(19) United States
(12) Patent Application Publication
(10) Pub. No.: US 2013/0172751 A1
(43) Pub. Date: Jul. 4, 2013

(54) SYSTEMS AND METHODS FOR SHOCK ABSORBING IN ULTRASOUND PROBES

(71) Applicants: Christian Heinrich, Zipf (AT); Christian Holl, Zipf (AT)

(72) Inventors: Christian Heinrich, Zipf (AT); Christian Holl, Zipf (AT)

(73) Assignee: GENERAL ELECTRIC COMPANY, Schenectady, NY (US)

(21) Appl. No.: 13/342,170

(22) Filed: Jan. 2, 2012

(51) Int. Cl. A61B 8/14

(52) U.S. Cl. 600/444; 600/447

(57) ABSTRACT

Methods and systems for shock absorbing in ultrasound probes are provided. One ultrasound probe has a housing and a scan head within the housing, wherein the scan head includes a transducer array. The ultrasound provide further includes an axle coupled to the scan head allowing rotation of the scan head and a shock absorbing member within the scan head coupled between the transducer array and the axle. The shock absorbing member is configured to allow relative movement between the axle and the transducer array.

### BACKGROUND OF THE INVENTION

The subject matter disclosed herein relates generally to ultrasound systems and, more particularly, to probes for ultrasound medical imaging systems.

Ultrasound systems typically include ultrasound scanning devices, such as ultrasound probes having different transducers that allow for performing various different ultrasound scans (e.g., different imaging of a volume or body). The ultrasound probes are typically connected to an ultrasound system that controls the operation of the probes. The probes include a scan head having a plurality of transducer elements (e.g., piezoelectric crystals), which may be arranged in an array. The ultrasound system drives the transducer elements within the array during operation, such as, during a scan of a volume or body, which may be controlled based upon the type of scan to be performed.

In mechanical volume probes, often referred to as mechanical four-dimensional (4D) probes, the scan head mechanically moves during scanning operation. In some probes, the scan head moves (e.g. rotates) in a sealed wet chamber having an acoustic membrane surrounding a scan head housing that contacts a patient during a scan. The wet chamber is typically filled with an acoustic liquid to allow acoustic coupling during scanning (e.g., during transmissions). In these probes, because the scan head has to move, the transducer array cannot be sealed within the plastic probe housing, such as encased in an epoxy. As a result, the components of the scan head, and especially the transducer array, are more susceptible to damage during impact, such as if the probe is dropped or sharply contacts another object. Thus, there is an increased likelihood that during day to day scanning operations, damage may occur, particularly to the transducer array, which would cause the probe to not operate or not operate properly. The probe would then have to be removed from service and repaired or replaced.

Thus, mechanical probes are more likely to be damaged by drop or impact events resulting in reliability issues. Additionally, increased costs in service swaps of the probes can result.

### BRIEF DESCRIPTION OF THE INVENTION

In one embodiment, an ultrasound probe is provided having a housing and a scan head within the housing, wherein the scan head includes a transducer array. The ultrasound probe further includes an axle coupled to the scan head allowing rotation of the scan head and a shock absorbing member within the scan head coupled between the transducer array and the axle. The shock absorbing member is configured to allow relative movement between the axle and the transducer array.

In another embodiment, an ultrasound probe is provided having a housing, a transducer array within the housing and a mechanically movable scan head supporting the transducer array and having an axle coupled thereto. The ultrasound probe further includes a shock absorber coupled within at least one side wall of the scan head, wherein the shock absorber is compressible to allow relative movement between the axle and the transducer array. The ultrasound probe also includes a motor for driving the axle to rotate the scan head.

In a further embodiment, a method for absorbing shock in an ultrasound probe is provided. The method includes providing probe a housing and positioning a scan head within the probe housing, wherein the scan head includes a transducer array and having an axle coupled to the scan head allowing rotation of the scan head. The method further includes coupling a shock absorbing member within the scan head between the transducer array and the axle, wherein the shock absorbing member is configured to allow relative movement between the axle and the transducer array.

### BRIEF DESCRIPTION OF THE DRAWINGS

FIG. 1 is a cross-sectional view of a portion of an ultrasound probe in accordance with one embodiment.

FIG. 2 is a perspective view of a portion of the ultrasound probe of FIG. 1 having the probe housing removed.

FIG. 3 is a perspective view of a portion of an ultrasound probe in accordance with another embodiment.

FIGS. 4-6 are diagrams an ultrasound probe in accordance with one embodiment showing a moving scan head.

FIG. 7 is a block diagram of an ultrasound system in accordance with one embodiment.

FIG. 8 is a block diagram of an ultrasound processor module of the ultrasound system of FIG. 7 formed in accordance with various embodiments.

### DETAILED DESCRIPTION

The following detailed description of various embodiments will be better understood when read in conjunction with the appended drawings. To the extent that the figures illustrate diagrams of structural or functional blocks of the various embodiments, the blocks are not necessarily indicative of the division between hardware or circuitry. Thus, for example, one or more of the blocks may be implemented in a single piece of hardware or multiple pieces of hardware. It should be understood that the various embodiments are not limited to the arrangements and instrumentality shown in the drawings.

Described herein are various embodiments for providing shock absorption (e.g., dampening of impact forces) for an ultrasound probe, particularly an ultrasound probe having a moving scan head. However, it should be noted that although the various embodiments are described in connection with a probe have a particular mechanical configuration, the shock absorption may be provided to different types and configurations of probes.

By practicing at least one embodiment, one or more components of an ultrasound probe, especially the transducer array in mechanical probes are supported to provide damage protection in the event of dropping of or impact to the ultrasound probe.

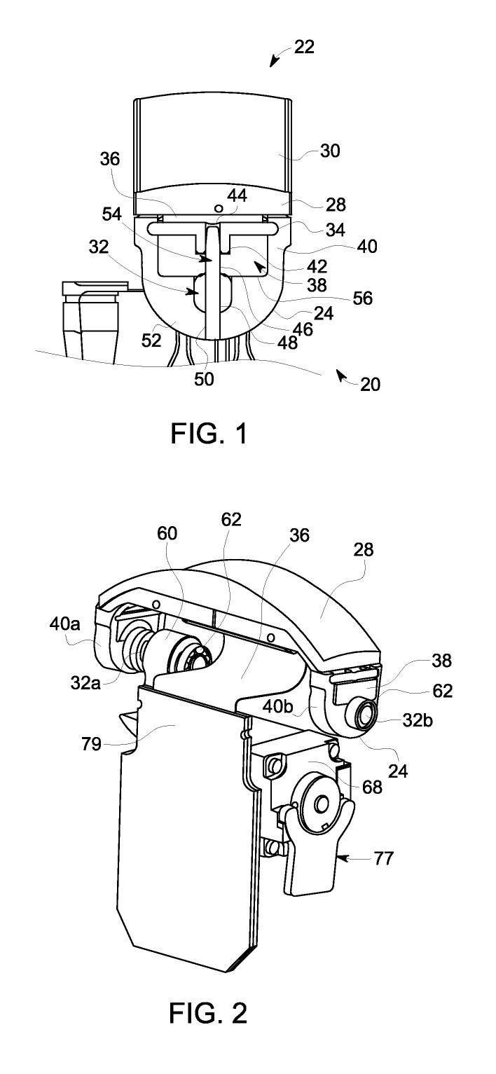

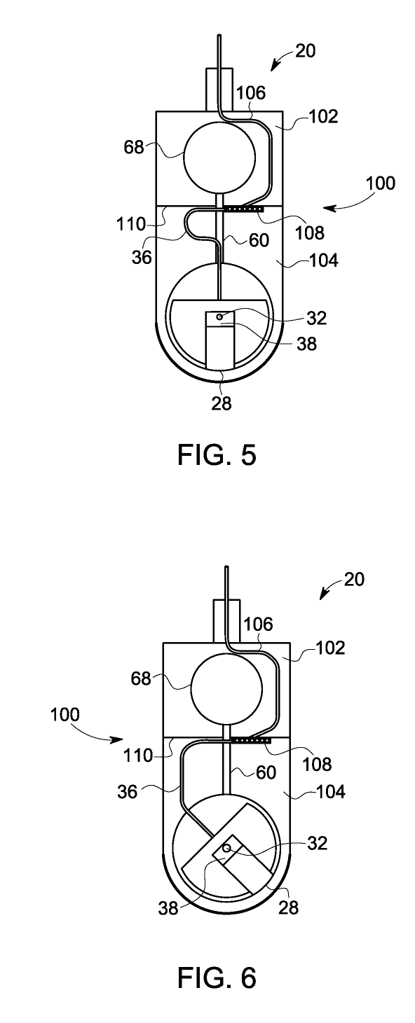

In particular, various embodiments provide an ultrasound probe 20, a portion of which, namely a scanning end 22, is shown in FIG. 1 illustrating a cross-sectional view. The ultrasound probe 20 in the illustrated embodiment is a volume imaging probe having a mechanically moving scan head 24 (which defines a transducer carrier or bridge for supporting a transducer array 28) within a scan head housing, such as a chamber 104 (shown in FIGS. 4-6). The scan head housing in one embodiment defines a wet chamber of the ultrasound probe 20 with a separate dry chamber having contained therein drive means for mechanically controlling (e.g., rotating) the scan head 24 to move the transducer array 28, which may be covered by a lens 30. Means for communicating with and electrically controlling the transducer array 28 are also provided as described in more detail herein in connection with FIG. 2, which may include one or more flexible printed circuit boards 36 (also referred to herein as flex PCBs).

It should be noted that although the transducer array 28 is shown as a curved array element, different configurations may be provided. For example, the transducer array 28 may be a linear array.

The scan head 24 may be in a chamber having an acoustic liquid therein and includes transducer driving means for moving (e.g., rotating) the transducer array 28 and transducer control means for selectively driving elements of the transducer array 28 (e.g., the piezoelectric ceramics of the transducer array 28). The transducer driving means generally includes a transducer axle 32 in connection with the scan head 24, for example, coupled to the scan head and extending within a drive shaft opening formed within the scan head 24. A connector support member 34 is also coupled within the scan head 24 for supporting the flex PCBs 36 in connection with the transducer array 28.

The scan head 24 generally defines a transducer carrier or transducer bridge, such that when the transducer axle 30 moves, in particular rotates, to move the scan head 24 movement of the transducer array 28 mounted thereto is also provided. It should be noted that the flex PCB 36 is coupled between the connector support member 34 and the transducer array 28, and is electrically connected to the transducer array 28.

A shock absorbing member 38 is positioned within the scan head 24 between the transducer array 28 and the transducer axle 32, and more particularly, between the connector support member 34 and the transducer axle 38. For example, in the illustrated embodiment, the shock absorbing member 38 is an elastic polymer (elastomer) shock absorber, such as a rubber shock absorber. However, it should be noted that the shock absorbing member 38 may be formed from different elastic and resilient materials or members, such as based on a desired or required amount of impact or shock resistance. For example, the shock absorbing member 38 in various embodiments may be formed from a thermoset or thermoplastic material. In some embodiments, the shock absorbing member 38 is formed from a material that deforms and then returns to an original shape (or substantially the original shape). However the shock absorbing member 38 may also be formed from any other material, for example, metal in some embodiments.

The shock absorbing member 38 is coupled within a side wall 40 of the scan head 24, such as within a cut-out region or slot that is sized and shaped to receive the shock absorbing member 38 therein. The shock absorbing member 38 may be permanently affixed within the scan head 24, for example, using a suitable adhesive. However, other coupling arrangements may be provided, such as a mechanical attachment (e.g., using a bracket) or an interference or snap-fit connection.

The shock absorbing member 38 is illustrated as a generally rectangular shaped member having a slot 42 on a top end for receiving a base of the connector support member 34, which may be permanently affixed therein, such as using a suitable adhesive. It should be noted that the size and shape of the shock absorbing member 38 may be varied as desired or needed. For example, the thickness or height of the shock absorbing member 38 may be increased or decreased to provide more or less resistance to a force applied thereto. In the illustrated embodiment, the shock absorbing member 38 generally defines a force dampening component provided as a block of material.

In one embodiment, the connector support member 34 has an opening 44 therethrough that is aligned with an opening 46 through the shock absorbing member 38 when coupled together. Additionally, the transducer axle 32 also includes an opening 48 therethrough that is aligned with the openings 44 and 46. An opening 50 is further provided through a base portion 52 of the scan head 24 and aligned with the openings 44, 46, and 48.

Thus, the openings 44, 46, 48 and 50 generally define a bore at the side wall 40 of the scan head 24. In the illustrated embodiment, a pin 54 is provided (e.g., inserted) within and through the openings 44, 46, 48 and 50 extending from the base portion 52 into the opening 44 of the connector support member 34. In this embodiment, the pin 54 is fixedly secured (e.g., adhesively coupled) within the opening 50 of the base 52

The pin 54 is not fixedly secured within the openings 44, 46 and 48 such that the transducer axle 32 is capable of movement relative to the connector support member 34 along the pin 54. For example, the transducer axle 32 may translate or slide along the pin 54 when the probe 22 is subjected to a force (e.g., a drop force) such that the shock absorbing member 38 is compressed between the connector support member 34 and the transducer axle 32 (e.g., the away from a base region 56 of the cut-out region or slot within the side wall 40). It should be noted that the openings 44, 46, 48 and 50 may define a bore have a defined size, for example, slightly larger than the diameter of the pin 54. In one embodiment, for example, the openings 44, 46, 48 and 50 have a diameter of about 1.5 millimeters (mm). However, other sizes for the openings 44, 46, 48 and 50 may be provided as desired or needed.

Thus, the connector support member 34 along with openings 46, 48 and 50 guides the pin 54 to increases the deceleration length of components within the probe 20, such as the transducer array 28. Accordingly, the deceleration value is reduced, such that an impact force is also reduced. The shock absorbing member 38 provides compliance to the more fragile or impact affected parts of the probe 20, which in the illustrated embodiment includes the transducer array 28. In various embodiments, the pin 54 transmit a torque from the transducer axle 32 to the connector support member 34 and allows compression of the shock absorbing member 38 to reduce a deceleration force. Thus, a force applied to the probe 20 (e.g., a drop force) may be dampened.

It should be noted that different configurations may be provided. For example, as shown in FIG. 2, the scan head 24 may be mounted to two separate transducer axles 32a and 32b that do not extend entirely between the side walls 40 of the scan head 24. In the illustrated embodiment, the transducer axle 32a extends about a third of the total distance between the side walls 40 and engages a gear arrangement 60, which in this embodiment is a toothed gear arrangement coupled to a motor 68.

However, other arrangements to drive the transducer axle 32a may be provide, for example, a ball drive arrangement or a two-stage gear arrangement having a belt drive and a rope drive. Additionally, a ball bearing 62 is provided in connection with the transducer axle 32a, which reduces rotational friction and supports radial and axial loads. In this embodiment, a ball bearing 62 is also provided in connection with the transducer axle 32b on an opposite side of the scan head 24. However, the transducer axle 32b extends within the side wall 40 of the scan head 24 and outward therefrom a distance sufficient to support the ball bearing 62. Accordingly, the transducer axle 32b is shorter than the transducer axle 32a.

The transducer array 28 is connected with one or more processing or control boards 79 via the flex PCBs 36. For example, the one or more processing or control boards 79 may be tuning and/or termination boards for the transducer array 28. However, any other type of processing or control board may be provided as desired or needed. Other components also may be provided in some embodiments. For example, in one embodiment, an alignment sensor 77 may be provided, which may be a Hall sensor PCB that operates to provide center position alignment of the transducer array 28.

It also should be appreciated that variations and modification are contemplated. For example, the transducer axle 32 may be a single axle that extends from one side wall 40a of the scan head 24 to the other side wall 40b of the scan head 24.

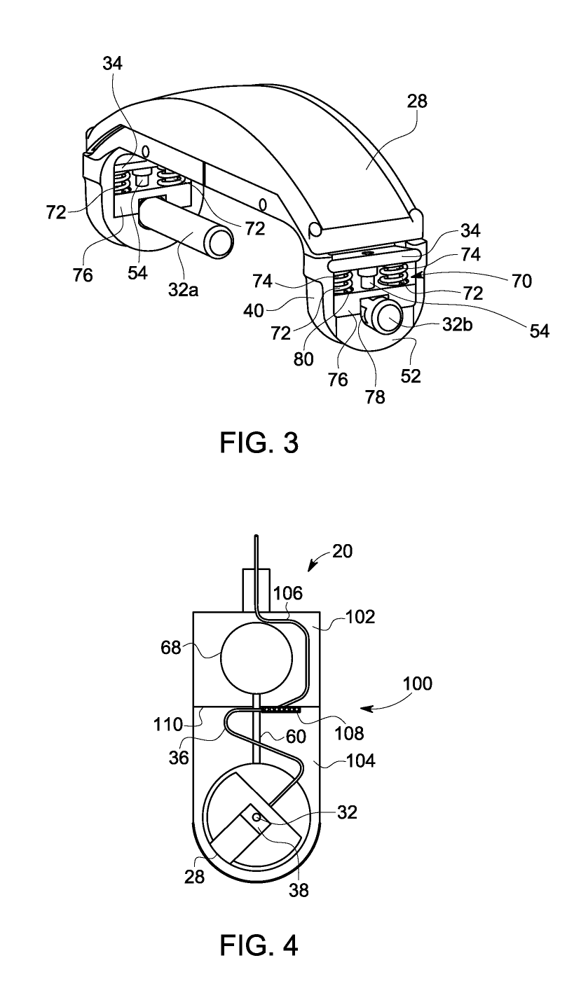

The shock absorbing member 38 also may be modified in various embodiments. For example, as shown in FIG. 3, the shock absorbing member 38 may be a spring arrangement 70 having a plurality of springs 72 (e.g., metal or plastic spiral springs), illustrated as two coil springs. However, different types of springs 72 and number of springs 72 may be provided as desired or needed. The springs 72 are mounted over rods 74 that extend between the connector support member 34 and a spring support member 76. The spring support member 76 is mounted within the cut-out or slot of the side wall 40 and includes an opening 78 for receiving therethrough a portion of the transducer axle 32b. The base 52 of the side wall 40 also includes an opening therethrough for receiving the transducer axle 32b, such that the transducer axle 32b can rotate as described herein. It should be noted that a similar arrangement is provided for the transducer axle 32a.

The spring support member 76 also includes openings 80 therein for receiving a portion of the springs 72. Accordingly, the springs 72 are aligned within the openings 80 and along the rods 74 such that the transducer axle 32b can move relative to the connector support member 34 along the pin 54 and the springs 72. Thus, the pin 54 transmits a torque from the transducer axle 32 to the connector support member 34 and allows compression of the springs 72 to reduce a deceleration force.

It should be noted that the spring constant of the springs 72 may be varied based on a desired or required amount of impact absorbing or resistance. Additionally, the length, width or number of coils of the springs 72 likewise may be varied.

It should be noted that in the various embodiments, the shock absorbing arrangements, although described in connection with one side of the scan head 24 may be similarly provided on the other side of the scan head 24.

FIGS. 4 through 6 illustrate one embodiment of the ultrasound probe 20 illustrating operation of the elements of a moving transducer array 28. In particular, these Figures illustrate the transducer array 28 in different rotational positions. The ultrasound probe 20 is a volume imagining probe that may be in communication with a host system. In one embodiment, the probe 20 includes a housing 100 having a first chamber 102 (e.g., a dry chamber) and a second chamber 104 (e.g., a wet chamber). The first chamber 102 and second chamber 104 may be formed as a single unit (e.g., unitary construction) or may be formed as separate units connected together (e.g. modular design). In an exemplary embodiment, the first chamber 102 is a dry or air chamber having contained therein drive means for mechanically controlling the transducer array 28 and communication means for electrically controlling the transducer array 28. The drive means generally includes the motor 68 (e.g., stepper motor) and the gear arrangement 60 (shown in FIG. 2). The communication means generally includes a system cable 106 connected to the flex PCBs 36 to communicate with the host system to drive the elements of the transducer array 28 (e.g., selectively activate the elements of the transducer array 28).

However, it should be noted that in some embodiments, only a single dry chamber is provided. It also should be noted that although the drive means and communication means are described herein having specific component parts, they are not so limited. For example, the drive means may have a different gear arrangement and the communication means may have different connection members or transmission lines.

In this exemplary embodiment, the second chamber 104 is a wet chamber (e.g., chamber having acoustic liquid therein) having contained therein transducer driving means for moving (e.g., rotating) the transducer array 28 and transducer control means for selectively driving elements of the transducer array 28 (e.g., the piezoelectric ceramics). The transducer driving means generally includes drive means as described herein and having the shock absorbing member 38.

The transducer control means generally includes a connection member 108 for interconnecting the system cable 106 and the flex PCBs 36 (e.g., four scan head flexible printed circuit boards) having one or more communication lines for providing communication therebetween. In one exemplary embodiment, the connection member 108 is formed from one or more rigid printed circuit boards that interconnect the system cable 106 and the flex PCBs 36 through a sealing member 110 (that provides a liquid tight seal between the first chamber 102 and the second chamber 104).

It should be noted that although the transducer driving means and transducer control means are described herein having specific component parts, these elements are not so limited. For example, the transducer driving means may have a different shaft arrangement and the transducer control means may have different control circuits or transmission lines. It also should be noted that additional or different component parts may be provided in connection with the probe 20 as needed or desired, and/or based upon the particular type and application of the probe 20. It further should be noted that the transducer array 28 may be configured for operation in different modes, such as, for example, a 1D, 1.25D, 1.5D, 1.75D, 2D, 3D and 4D modes of operation.

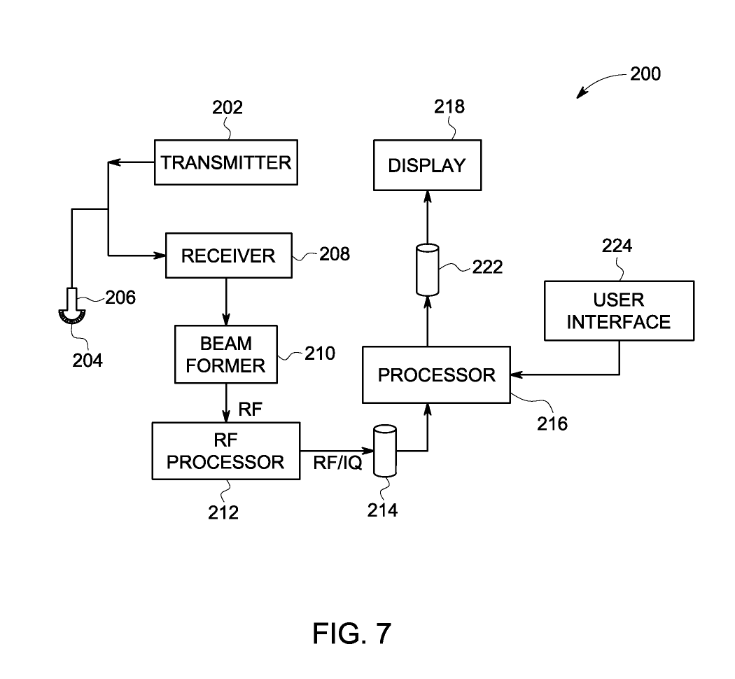

The various embodiments described herein may be implemented in connection with an imaging system shown in FIG. 7. Specifically, FIG. 7 illustrates a block diagram of an exemplary ultrasound system 200 that is formed in accordance with various embodiments. The ultrasound system 200 includes a transmitter 202, which drives a plurality of transducers 204 within an ultrasound probe 206 to emit pulsed ultrasonic signals into a body. A variety of geometries may be used. For example, the probe 206 may be used to acquire 2D, 3D, or 4D ultrasonic data, and may have further capabilities such as 3D beam steering. Other types of probes 206 may be used. The probe 206 also may be embodied as the probe 20 described herein having a shock absorber. The ultrasonic signals are back-scattered from structures in the body, like blood cells or muscular tissue, to produce echoes which return to the transducers 204. The echoes are received by a receiver 208. The received echoes are passed through a beamformer 210, which performs beamforming and outputs an RF signal. The beamformer may also process 2D, 3D and 4D ultrasonic data. The RF signal then passes through an RF processor 212. Alternatively, the RF processor 212 may include a complex demodulator (not shown) that demodulates the RF signal to form IQ data pairs representative of the echo signals. The RF or IQ signal data may then be routed directly to RF/IQ buffer 214 for temporary storage.

The ultrasound system 200 also includes a signal processor 216. The signal processor 216 processes the acquired ultrasound information (i.e., RF signal data or IQ data pairs) and prepares frames of ultrasound information for display on a display 218. The signal processor 216 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 214 during a scanning session and processed in less than real-time in a live or off-line operation. A user interface, such as user interface 224, allows an operator to enter data, enter and change scanning parameters, access protocols, select image slices, and the like. The user interface 224 may be a rotating knob, switch, keyboard keys, mouse, touch screen, light pen, or any other suitable interface device.

The ultrasound system 200 may continuously acquire ultrasound information at a frame rate that exceeds 50 frames per second—the approximate perception rate of the human eye. The acquired ultrasound information, which may be the 3D volume dataset, is displayed on the display 218. The ultrasound information may be displayed as B-mode images, M-mode, volumes of data (3D), volumes of data over time (4D), or other desired representation. An image buffer (e.g., memory) 222 is included for storing processed frames of acquired ultrasound information that are not scheduled to be displayed immediately. The image buffer 222 in one embodiment is of sufficient capacity to store at least several seconds worth of frames of ultrasound information. The frames of ultrasound information are stored in a manner to facilitate retrieval thereof according to its order or time of acquisition. The image buffer 222 may comprise any known data storage medium.

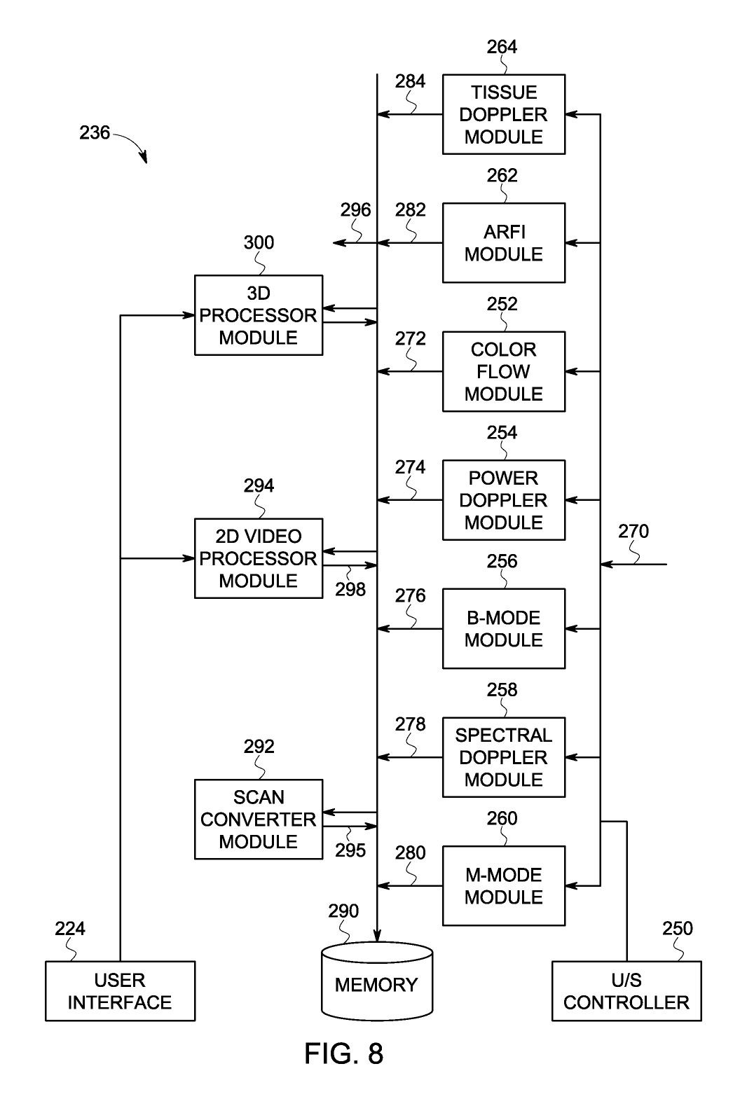

FIG. 8 illustrates an exemplary block diagram of an ultrasound processor module 236, which may be embodied as the signal processor 216 of FIG. 7 or a portion thereof. The ultrasound processor module 236 is illustrated conceptually as a collection of sub-modules, but may be implemented utilizing any combination of dedicated hardware boards, DSPs, processors, etc. Alternatively, the sub-modules of FIG. 8 may be implemented utilizing an off-the-shelf PC with a single processor or multiple processors, with the functional operations distributed between the processors. As a further option, the sub-modules of FIG. 8 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 8 may be controlled by a local ultrasound controller 250 or by the processor module 236. The sub-modules 252-264 perform, for example, mid-processor operations. The ultrasound processor module 236 may receive ultrasound data 270 in one of several forms. In the embodiment of FIG. 8, the received ultrasound data 270 constitutes I,Q data pairs representing the real and imaginary components associated with each data sample. The I,Q data pairs are provided to one or more of a color-flow sub-module 252, a power Doppler sub-module 254, a B-mode sub-module 256, a spectral Doppler sub-module 258 and an M-mode sub-module 260. Optionally, other sub-modules may be included such as an Acoustic Radiation Force Impulse (ARFI) sub-module 262 and a Tissue Doppler (TDE) sub-module 264, among others.

Each of sub-modules 252-264 are configured to process the I,Q data pairs in a corresponding manner to generate color-flow data 272, power Doppler data 274, B-mode data 276, spectral Doppler data 278, M-mode data 280, ARFI data 282, and tissue Doppler data 284, all of which may be stored in a memory 290 (or memory 214 or memory 222 shown in FIG. 7) temporarily before subsequent processing. For example, the B-mode sub-module 256 may generate B-mode data 276 including a plurality of B-mode image planes, such as in a biplane or triplane image acquisition as described in more detail herein.

The data 272-284 may be stored, for example, as sets of vector data values, where each set defines an individual ultrasound image frame. The vector data values are generally organized based on the polar coordinate system.

A scan converter sub-module 292 accesses and obtains from the memory 290 the vector data values associated with an image frame and converts the set of vector data values to Cartesian coordinates to generate an ultrasound image frame 295 formatted for display. The ultrasound image frames 295 generated by the scan converter module 292 may be provided back to the memory 290 for subsequent processing or may be provided to the memory 214 or the memory 222 (both shown in FIG. 7).

Once the scan converter sub-module 292 generates the ultrasound image frames 295 associated with, for example, B-mode image data, and the like, the image frames may be restored in the memory 290 or communicated over a bus 296 to a database (not shown), the memory 214, the memory 214 and/or to other processors.

The scan converted data may be converted into an X,Y format for video display to produce ultrasound image frames. The scan converted ultrasound image frames are provided to a display controller (not shown) that may include a video processor that maps the video to a gray-scale mapping for video display. The gray-scale map may represent a transfer function of the raw image data to displayed gray levels. Once the video data is mapped to the gray-scale values, the display controller controls the display 218 (shown in FIG. 7), which may include one or more monitors or windows of the display, to display the image frame. The image displayed in the display 218 is produced from image frames of data in which each datum indicates the intensity or brightness of a respective pixel in the display.

Referring again to FIG. 8, a 2D video processor sub-module 294 combines one or more of the frames generated from the different types of ultrasound information. For example, the 2D video processor sub-module 294 may combine a different image frames by mapping one type of data to a gray map and mapping the other type of data to a color map for video display. In the final displayed image, color pixel data may be superimposed on the gray scale pixel data to form a single multi-mode image frame 298 (e.g., functional image) that is again re-stored in the memory 290 or communicated over the bus 296. Successive frames of images may be stored as a cine loop in the memory 290 or memory 214 (shown in FIG. 7). The cine loop represents a first in, first out circular image buffer to capture image data that is displayed to the user. The user may freeze the cine loop by entering a freeze command at the user interface 224. The user interface 224 may include, for example, a keyboard and mouse and all other input controls associated with inputting information into the ultrasound system 200 (shown in FIG. 7).

A 3D processor sub-module 300 is also controlled by the user interface 224 and accesses the memory 290 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

The ultrasound system 200 of FIG. 7 may be embodied in a small-sized system, such as laptop computer or pocket sized system as well as in a larger console-type system.

Thus, various embodiments provide an ultrasound probe having a shock absorber. It should be noted that the shock absorbing arrangement may be located in different portions of the probe. For example, shock absorption in accordance with one or more embodiments may be implemented in other probe designs, such as by inverting the arrangement to provide shock compliance to other heavier probe components (e.g., the electronics) instead of the transducer array and probe housing. Accordingly, the impact force on the transducer/housing component are also reduced, thereby increasing drop/impact reliability.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive, optical disk drive, and the like. The storage device may also be other similar means for loading computer programs or other instructions into the computer or processor.

As used herein, the term “computer” or “module” may include any processor-based or microprocessor-based system including systems using microcontrollers, Reduced Instruction Set Computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. An ultrasound probe comprising:
a housing;
a scan head within the housing, the scan head including a transducer array;
an axle coupled to the scan head allowing rotation of the scan head; and
a shock absorbing member within the scan head coupled between the transducer array and the axle, the shock absorbing member configured to allow relative movement between the axle and the transducer array.

2. The ultrasound probe of claim 1, wherein the shock absorbing member comprises an elastomer material within a side wall of the scan head.

3. The ultrasound probe of claim 2, wherein the elastomer material and the axle each have an opening therethough, the openings being aligned, and further comprising a pin extending through the openings allowing sliding movement of the axle relative to the transducer array along the pin.

4. The ultrasound probe of claim 3, wherein the pin is configured to allow compression of the elastomer material between the transducer array and the axle.

5. The ultrasound probe of claim 1, wherein the shock absorbing member comprises at least one spring within a side wall of the scan head.

6. The ultrasound probe of claim 1, further comprising a connector support member supporting a connector coupled to the transducer array and wherein the shock absorbing member is coupled between the connector support member and the axle.

7. The ultrasound probe of claim 6, wherein the shock absorbing member and the axle each have an opening therethough, the opening being aligned, and further comprising a pin extending through the openings allowing sliding movement of the axle relative to the transducer array along the pin, wherein the connector support member having an opening therethrough to guide the pin.

8. The ultrasound probe of claim 6, further wherein the connector is a flexible printed circuit board coupled between the connector support member and the transducer array.

9. The ultrasound probe of claim 1, wherein the shock absorbing member comprises a force dampening component within at least one side wall of the scan head.

10. The ultrasound probe of claim 9, wherein the force dampening component comprises one of a rubber material or a metal material.

11. The ultrasound probe of claim 9, wherein the force dampening component comprises a block of dampening material.

12. An ultrasound probe comprising:
a housing;
a transducer array within the housing;
a mechanically movable scan head supporting the transducer array and having an axle coupled thereto;
a shock absorber coupled within at least one side wall of the scan head, the shock absorber compressible to allow relative movement between the axle and the transducer array; and
a motor for driving the axle to rotate the scan head.

13. The ultrasound probe of claim 12, wherein the shock absorber is formed from an elastomer.

14. The ultrasound probe of claim 12, wherein the shock absorber comprises at least one spring.

15. The ultrasound probe of claim 12, further comprising a pin extending through the shock absorber and axle, the pin allowing the relative movement between the axle and the transducer array.

16. The ultrasound probe of claim 12, wherein the transducer array is operable in a four-dimensional (4D) imaging mode.

17. The ultrasound probe of claim 12, wherein the axle comprises two separate axles coupled to different side walls of the scan head.

18. The ultrasound probe of claim 12, wherein the axle is configured to move towards the transducer array when subjected to a force.

19. The ultrasound probe of claim 12, wherein the housing comprises a wet chamber and a dry chamber, the transducer array being in the wet chamber.

20. A method for absorbing shock in an ultrasound probe, the method comprising:
providing probe a housing;
positioning a scan head within the probe housing, the scan head including a transducer array and having an axle coupled to the scan head allowing rotation of the scan head; and
coupling a shock absorbing member within the scan head between the transducer array and the axle, the shock absorbing member configured to allow relative movement between the axle and the transducer array.
